(19) United States
(12) Patent Application Publication
(10) Pub. No.: US 2008/0138841 A1
(43) Pub. Date: Jun. 12, 2008

(54) Monitoring System For Sensing Microorganisms

(76) Inventors: Alfred Michael Vegvary, Western Australia (AU); Tiffany L. Atkinson, Western Australia (AU)

Correspondence Address: JAMES C. WRAY, 1493 CHAIN BRIDGE ROAD, SUITE 300, MCLEAN, VA, 22101, US

(21) Appl. No.: 11/884,968

(22) PCT Filed: Feb. 23, 2006
(86) PCT No.: PCT/AU2006/000231

§ 371 Date: Nov. 30, 2007

(30) Foreign Application Priority Data
Feb. 23, 2005 (AU) 2005900826

(51) Int. Cl. C12Q 1/04; G01N 21/62; C12M 1/34

(52) U.S. Cl. 435/7.32; 435/29; 436/172; 250/461.2

(57) ABSTRACT

A monitoring system comprising a sensing means operable to sense a prescribed microorganism in a prescribed environment and a human interfacing means, wherein the sensing means comprises means for emitting UV light and measuring resulting emissions from microorganisms, and the human interfacing means is operatively coupled to the sensing means and operable to generate an alert in response to the sensing means sensing a prescribed microorganism.

### FIELD OF THE INVENTION

The present invention relates to a monitoring system. In particular, the present invention relates to a monitoring system for the detection of microorganisms in food storage units

### BACKGROUND ART

With the frequent occurrences of food-borne illnesses being a wide spread problem throughout the world, various developments have been devised for early detection and control of food-borne bacteria. Measures and protocols are constantly updated to prevent contaminated products from reaching the market. Such protocols have been designed to lower costs associated with product recall or medical related factors. However, these methods of detection and control pertain directly to the manufacturing and commercial areas of the food chain. Precautions, safety procedures and protocols, monitoring and detection are well established in the food supply industry and are constantly being improved in order to control bacterial associated factors, like temperature regularity and cross-contamination.

Food safety strategies currently rely heavily upon end product testing to ensure the quality of the product prior to its release in the marketplace. However, this is not a guarantee that the consumable will not be contaminated when it reaches the consumers, due to handling, storage and marketplace conditions, bacterial transference and various other factors that are difficult to control and monitor.

There is a need for a system to detect food-borne bacteria once consumables have left the commercial line and are in a consumer's possession. Bacterial contamination is common and difficult to manage without education and knowledge of consumables and their potential risk factors (i.e. bacterial food borne illnesses associated with that product).

Throughout the specification, unless the context requires otherwise, the word “comprise” or variations such as “comprises” or “comprising”, will be understood to imply the inclusion of a stated integer or group of integers but not the exclusion of any other integer or group of integers.

### DISCLOSURE OF THE INVENTION

In accordance with the present invention, there is provided a monitoring system comprising a sensing means operable to sense a prescribed microorganism in a prescribed environment and human interfacing means operatively coupled to the sensing means and operable to generate an alert in response to the sensing means sensing a prescribed microorganism.

The monitoring system preferably further comprises a computer, operatively connected to the sensing means and operatively connected to the human interfacing means, wherein the computer comprises a database stored in a memory, a program stored in the memory and a processing means. The computer is operable to execute the program to enable the computer to perform various functions.

Preferably, the prescribed environment is a food storage unit. Preferably, the food storage unit is adapted to keep food cold and may be selected from the group comprising refrigerators, cool rooms, storage facilities, food transportation vehicles and eskies.

Advantageously, the present invention enables the risk management of food-borne bacteria to be extended to a user's refrigeration unit introducing a means for the user to control food-borne illnesses. The invention may be employed domestically as well as by various businesses and organisations thereby introducing food-borne bacterial management to restaurants, airlines, luxury transport services, cruise liners and other businesses commonly affected by illnesses brought about by consumption of contaminated food products.

Preferably, the prescribed microorganism is a plurality of microorganisms.

More preferably, the microorganisms are food-borne bacteria. It will be appreciated that there are an extremely large number of known food-borne bacteria. In particular, those food-borne bacteria that can have deleterious affects on humans and animals. Examples of such bacteria include Campylobacteria, Salmonella, Escherichia. Coli (E. Coli), Listeria and Shigella.

Campylobacter are a genus of bacteria, one species of which C. jejuni is a curved, rod-shaped bacterium. Salmonella are a genus of rod-shaped enterobacteria about 2 to 3 mm in diameter. There are two species within the genus, S. bongori and S. enterica which is divided into six subspecies. E. Coli are a species of rod-shaped bacteria. Listeria is a bacterial genus containing six species which are typified by Listeria monocytogenes, Shigella are rod-shaped bacteria.

The sensing means preferably comprises means for emitting UV light. Preferably the UV light has a wavelength of between about 260 nm and about 360 nm. More preferably, the UV light has a wavelength of between about 260 nm and about 280 nm. The sensing means preferably further comprises means for measuring emissions from microorganisms. Said emissions may be selected from fluorescence, luminescence including bioluminescence and chemiluminescence, laser scattering and reflection and refraction of light. Preferably, fluorescence emissions are measured. Preferably, the means for measuring fluorescence emission is provided in the form of a photodiode.

Without being limited by theory, it is believed that irradiation of a bacteria with UV light results in an emission spectrum that may be characteristic for a particular genus or species of microorganism. Further, information on the shape and size of the microorganism may be gleaned from the emission spectrum

The database preferably comprises information on food-borne microorganisms such as sizes, shapes and fluorescence characteristics.

Preferably, there is provided a plurality of sensing means.

Preferably, the plurality of sensing means are located inside the food storage unit.

The plurality of sensing means, the human interfacing means and the computer may utilise any power source known in the art, including but not limited to mains power and battery power. Preferably, each sensor comprises its own independent power source such as a battery.

The human interfacing means provides means by which the presence or absence of microorganisms may be indicated to a user of the food storage unit. The human interfacing means may be provided in the form of a visual indicator and/or an audible indicator. Preferably, both a visual indicator and an audible indicator are provided.

A visual indicator may be provided in any form known in the art including an LED unit and LCD display unit. Where the visual indicator is provided in the form of an LED unit, the presence of microorganisms in the food storage unit may be represented by a solid light or a flashing light. Where the visual indicator is provided in the form of an LCD display unit, the presence of microorganisms in the food storage unit may be represented by a solid light or a flashing light or a series of words or a signal on the LCD display unit.

Where provided, the LCD display unit may provide further information about the proposed location of the microorganisms.

Where the human interfacing means is provided in the form of an audible indicator, the presence of microorganisms in the food storage unit may be represented by an alarm.

Where the food storage unit is a refrigerator, there are preferably provided four sensors on each shelf and in each compartment of the refrigerator.

### BRIEF DESCRIPTION OF THE DRAWINGS

FIG. 1 is a schematic drawing of a monitoring system in accordance with the present invention; and

FIG. 2 is a drawing of a refrigerator with one side cutaway comprising a monitoring system in accordance with the present invention.

### BEST MODE(S) FOR CARRYING OUT THE INVENTION

Those skilled in the art will appreciate that the invention described herein is susceptible to variations and modifications other than those specifically described.

It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiment described herein, which is intended for the purpose of exemplification only. Functionally equivalent products, compositions and methods are clearly within the scope of the invention as described herein.

By way of example, the system of the present invention is described with reference to a domestic refrigerator, although such should not be seen as limiting the generality of the foregoing description.

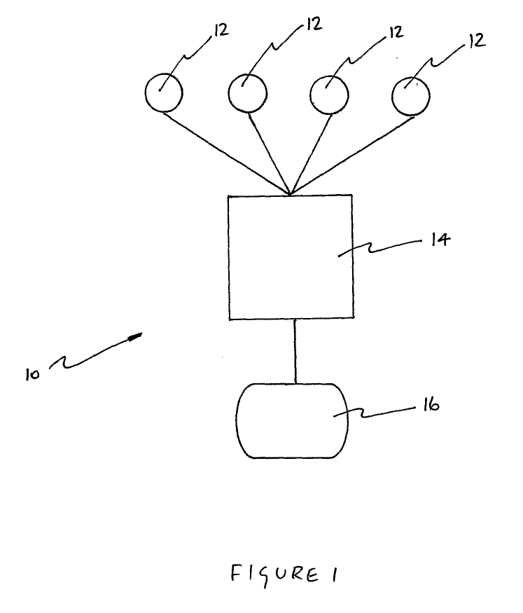

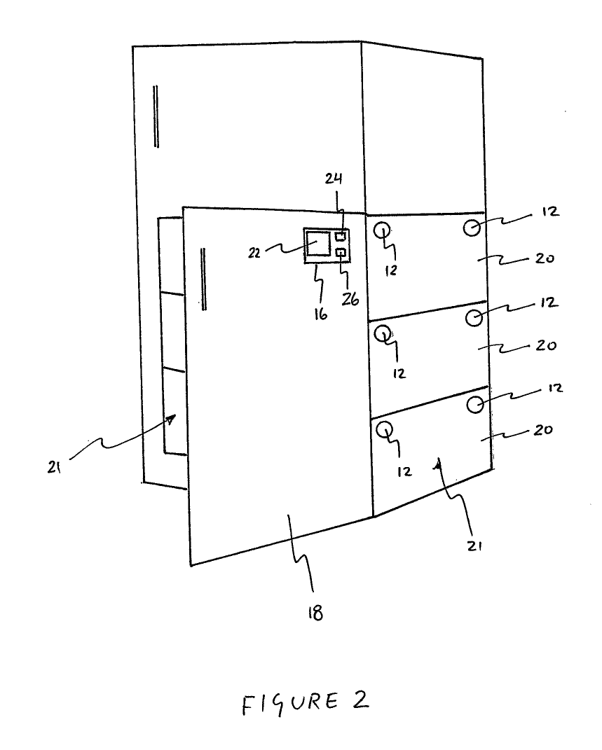

In accordance with the present invention, best seen in FIG. 1, the monitoring system 10 comprises a plurality of sensing means 12, a computer 14 operatively coupled to the plurality of sensing means 12 and a human interfacing means 16 operatively coupled to the computer 14. The plurality of sensing means 12 are located inside the refrigerator 18 and the computer 14 and the human interfacing means 16 are located on the exterior of the refrigerator 18, best seen in FIG. 2.

The system's multiple sensing means 12, located on the interior of the refrigerator 18, are strategically placed for maximum detection and results. It will be appreciated that the number of sensing means 12 will depend on the size of the refrigerator 18 and on the intensity and sensitivity of the sensing means 12. It is expected that in a standard family-sized fridge, about four sensing means will be required on each shelf and compartment such as a crisper. In FIG. 2, two sensing means 20 may be seen on each shelf of the cutaway portion of the refrigerator 18 with a side panel removed exposing the interior 21 of the refrigerator 18. The two remaining sensing means on each shelf cannot be seen.

Each sensing means 12 is operatively coupled to the computer 14 by any means known in the art including wired and wireless technologies.

The computer 14 comprises a database stored in a memory, a program and a processing means. The computer 14 is operable to execute the program stored in the memory to enable the computer 14 to perform various functions.

The human interfacing means 16 comprises an LCD screen 22, an alarm 24 and a reset button 26.

The sensing means are provided in the form of biosensors and are enclosed in a hard casing designed to protect the internal components of the sensor from the conditions within the refrigeration unit itself.

Each of the sensors comprise dual UV emitters in the form of pulsed or near UV lasers, although it is envisaged that other light sources such as UV laser diodes may be employed.

The light emitters should be sensitive enough to detect the smallest of particle with variable wavelengths. Investigations have shown that the most effective wavelength for bacterial fluorescence is between 260 nm and 360 nm. The shorter the wavelength, the higher the energy and therefore an increase in the fluorescence intensity.

Each sensor emits UV light at a specific wavelength which scans the environment and the sensor then records various measurements. The parameters to be measured for characterisation include particle size, shape, concentration, and multi-point angular measurements of the fluorescent light scattering. The fluorescence will be read by means of an inbuilt photodiode in the sensor itself. The multi-point angular readings allow for a variety of measurements to be taken, this feature will give multiple measurements for each particle which will be used by the software to more accurately calculate the shape and size of the particle, increasing the specificity of the determination of the particle, and draw a more accurate conclusion as to the species of the bacteria. The measurements are recorded and the data transmitted to the computer.

Each sensor is fitted with an inbuilt chip, programmed with algorithms to adjust the wavelength and intensity of the UV light emitted. For example, it is envisaged that across the range 260 nm to 280 nm, each sensor will emit a band of light at 260 nm and measurements recorded. Each sensor will then emit another band of light at a slightly higher wavelength and measurements recorded. The process will continue until the spectrum from 260 nm to 280 nm is covered. The varying wavelengths allow each particle's fluorescent scatter and intensity to be measured and therefore discriminating more accurately biological from interferants.

The chip in each sensor will control the transmissions of the data to the computer, and execute commands sent from the computer relating to scheduled in-sync readings, queued transmitting times, and instructions to make adjustments on how the readings are taken i.e. wavelength, intensity, power, etc. The computer will also relay information to delay a periodic reading due to interference i.e. an open refrigerator door. Other examples of elements that may cause interference, inaccurate readings, or component damage include, but are not limited to, condensation and/or bacterial or fungal growth on or near the internal sensor components.

On determination of the presence of a microorganism in the refrigerator, the computer compares the measurements to the information in the database to determine the nature of the microorganism.

It should be appreciated that depending on the nature of the particles measured for, infra red light or light from other regions of the electromagnetic spectrum may be utilised.

Periodic readings are processed and comparisons are made to rule out backgrounds. The computer can relay information to the sensors to delay a periodic reading due to interference such as an open refrigerator door, which is determined by a sensor on the refrigerator door. This is advantageous because of the dramatic change in conditions when the door is open as opposed to closed. An open door allows the atmosphere to be altered significantly; interferences to the readings are made present in the form of additional light and bacteria from the outside. Furthermore, the light that enters a fridge when the door is opened decreases the intensity of fluorescence emissions. Once the door has been closed the main computer resets the schedule for the sensors to take readings at a time that suitably allows for the atmosphere to restabilise.

The computer includes an onboard memory, for storing data and information to be used for determining bacterial existence, and differentiating bacteria from other common aerosols. A library of information pertaining to the bacteria to be detected is located in a database that allows for filtering, data matching, and identification. The library may be included in the software prior to installing the system into the refrigeration unit and holds key information about the bacteria, for example wavelengths, sizes, ranges, fluorescence, intensities, shape, and other identifying factors. Once data readings have been received from the sensors they are matched against the library for common characteristic that lead to determining the bacteria.

The library may be updatable via a direct link using a USB connection to a home or office computer with updates downloaded from the internet. Means of communication need not be limited to using only USB. A wireless network card may be incorporated into the computer, enabling it to be programmed to connect to the internet and update itself regularly. The updates will consist mainly of library bacterial updates. New bacteria are regularly discovered and with each new strain, a new set of parameters must be included in the database so that the parameters assessed by the sensors can be matched to these new bacteria.

The computer is specifically programmed with software that runs algorithms and diagnostics, filtering processes, data and sample matching, and information and library updating. Bacteria particles have different sizes, shapes and fluorescence properties. These parameters are integrated into the calculations and algorithms defining the information needed to make a diagnosis. Once the final figures are produced these are then run through the database and compared, matched and filtered in order to make an accurate assessment of the bacteria found. The end result is either a positive or negative determination for bacterial presence. A positive determination is displayed on the LCD screen and the alarm sounds periodically until the user acknowledges the message. This acknowledgement is registered via a reset button on the computer face, beside the LCD panel, and the alarm will cease.

When a threat is determined and identified, information is displayed, the alarm is sounded, but at all other times the display can save power by waiting in stand-by mode or displaying a screensaver. Alternatively, the display could display all outcomes as they are processed, displaying an “All Clear” message when the results are negative to bacteria. When the results are positive for bacteria, the display shows simple information so that the consumer can understand and take further action. This information includes the bacteria detected, level of risk associated with concentration levels, the section it was located in, and foods that are at risk and associated to the bacteria present, for example “Salmonella, high risk, lower level, chicken”.

Information of the positive determination is displayed in a language the consumer can understand clearly so that appropriate action can be taken.

Intermittent regular readings are processed and used as a comparative against all readings taken over a specific timeframe (i.e. readings taken every 10 min over a 1-2 hr timeframe) to establish an atmospheric aerosol background. The implementation of neural networks defines systematic changes, in harmony with the algorithms, to identify even the most minor of characteristic aerosol differences. This decreases the chance of a false alarm due to the complexity of the atmospheric background by eliminating elements of commonality in the previous readings.

Other functions for the computer comprise controlling the network system itself. This includes synchronisation of the readings by transmitting queue information, countdown sequences, initiating sequences, delays and response times to a sensor's chip. This allows for synchronised scanning and controls the influx of data being received from the sensors at any given time. Although the readings are taken simultaneously, the data is relayed back to the main computer one sensor at a time, analysed and saved. Upon finalising the data processing for each sensor, comparisons are made, and the process of determination can be initiated, processed and displayed if positive identification of bacterium is made.

Applications in areas of security and threat detection are feasible by utilising the system to detect biological threats in any enclosed atmosphere. For example, devices at customs, quarantine, airport security, and anywhere that could utilise a resource that detects biological aerosols as a means to deterring infestation.

The system may further be utilised in the medical industry to detect biological threats prior to infection on a grand scale, both in and around consumables or operating theatres.

1. A monitoring system comprising a sensing means operable to sense a prescribed microorganism in a prescribed environment and human interfacing means operatively coupled to the sensing means and operable to generate an alert in response to the sensing means sensing a prescribed microorganism.

2. A monitoring system according to claim 1, wherein the system comprises a computer, operatively connected to the sensing means and operatively connected to the human interfacing means.

3. A monitoring system according to claim 1, wherein the computer comprises a database stored in a memory, a program stored in the memory and a processing means.

4. A monitoring system according to claim 1, wherein the prescribed environment is a food storage unit.

5. A monitoring system according to claim 4, wherein the food storage unit is a refrigerator, a cool rooms, a food storage facility, a food transportation vehicle or an esky.

6. A monitoring system according to claim 1, wherein the prescribed microorganism is a plurality of microorganisms.

7. A monitoring system according to claim 7, wherein the microorganisms are food-borne bacteria.

8. A monitoring system according to claim 1, wherein the sensing means comprise means for emitting UV light.

9. A monitoring system according to claim 8, wherein the UV light has a wavelength of between about 260 nm and about 360 nm.

10. A monitoring system according to claim 8, wherein the UV light has a wavelength of between about 260 nm and about 280 nm.

11. A monitoring system according to claim 1, wherein the sensing means comprises means for measuring emissions from microorganisms.

12. A monitoring system according to claim 11, wherein the emissions are selected from fluorescence, luminescence including bioluminescence and chemiluminescence, laser scattering and reflection and refraction of light.

13. A monitoring system according to claim 12, wherein the means for measuring fluorescence emission is provided in the form of a photodiode.

14. A monitoring system according to claim 3, wherein the database comprises information on food-borne microorganisms such as sizes, shapes and fluorescence characteristics.

15. A monitoring system according to claim 1, wherein there is provided a plurality of sensing means.

16. A monitoring system according to claim 1, wherein the sensing means is located inside the food storage unit.

17. A monitoring system according to claim 1, wherein the human interfacing means is a visual indicator.

18. A monitoring system according to claim 1, wherein the human interfacing means is an audible indicator.

19. A monitoring system according to claim 1, wherein the human interfacing means is both a visual indicator and an audible indicator.

20. A monitoring system according to claim 17, wherein the visual indicator is an LED unit and/or LCD display unit.

21. A refrigerator comprising a monitoring system in accordance with claim 1.

22. (canceled)
